(19) United States
(12) Patent Application Publication
(10) Pub. No.: US 2016/0075951 A1
(43) Pub. Date: Mar. 17, 2016

(54) METHODS FOR PRODUCTION OF LIQUID HYDROCARBONS FROM METHANE AND CO2

(71) Applicant: Gunnar SANNER, Stathelle (NO)

(72) Inventor: Gunnar SANNER, Stathelle (NO)

(21) Appl. No.: 14/785,051

(22) PCT Filed: Apr. 10, 2014
(86) PCT No.: PCT/EP2014/057272

§ 371 Date: Oct. 16, 2015

(30) Foreign Application Priority Data
Apr. 19, 2013 (NO) 20130542

(51) Int. Cl. C10G 2/00; C01B 3/34; C07C 29/151

(52) U.S. Cl. CPC C10G 2/32; C07C 29/1518; C01B 3/34; C01B 2203/062; C01B 2203/1241; C01B 2203/0216; C01B 2203/84

(57) ABSTRACT

A liquid hydrocarbon production method comprising a) reacting methane with water to form syngas containing hydrogen, b) reacting a part of the hydrogen with carbon dioxide to form methane and water, c) supplying said methane and water obtained from carbon dioxide to the syngas forming reaction and d) reacting the remaining syngas to form liquid hydrocarbons is dis closed.

The present invention relates to methods for production of hydrocarbons from methane and CO₂. Especially the present invention relates to integrated methods for transforming methane gas and CO₂ to liquid hydrocarbons applicable for use as fuel or for other purposes.

### BACKGROUND

The transformation of methane to liquid hydrocarbons is generally known as Gas-to-Liquid (GTL). The main purpose of these reactions is as the name indicates to transform natural gas/methane to hydrocarbons that are liquid at room temperature.

These liquid hydrocarbons are more compact energy carriers, easier to handle than gas and are also applicable as raw materials for other processes such as production of polymers.

Different processes are known for performing GTL today. The main principle of the existing GTL plants is the reaction of methane with oxygen. During the reaction part of the methane will be full oxidised providing heat for the transformation process.

### PRIOR ART

Known processes for preparation of syngas and formation of methanol and other hydrocarbons are for instance disclosed in US2007/0004809 and WO02/38699.

Paul Sabatier originally discovered the influence of a nickel catalyst on the reaction between carbon dioxide and hydrogen and has given name to the process of reacting CO₂ and hydrogen to receive methane and H₂O.

WO03/048034 discloses a process wherein methane is reacted with steam to generate a carbon monoxide and hydrogen gas mixture. This mixture is then used in a Fisher-Tropsch synthesis to prepare liquid hydrocarbons.

EP2371799 discloses a methanol synthesis method using mixed reforming of natural gas and carbon dioxide. The method is limited to the production of methanol.

US2008319093 discloses a method of producing methanol or dimethyl ether, from reformation of natural gas and reaction with carbon dioxide.

### Objectives of the Invention

The objective of the present invention is to provide an improved method for transforming gas to liquid.

A further objective is to provide a method with increased cost efficiency and increased outcome of liquid hydrocarbons.

Yet another objective of the present invention is to provide a method which can be performed with thermal energy as the additional energy input, more preferably with sustainable energy as the additional energy input.

It is an aim to provide an energy efficient process. The present provides a liquid hydrocarbon production method comprising
1a) reacting methane with H₂O to form syngas containing hydrogen and carbon monoxide,
1b) reacting a part of the hydrogen with carbon dioxide to form methane and H₂O,
1c) supplying said methane and H₂O obtained from carbon dioxide to the syngas forming reaction and
1d) reacting the remaining syngas to form liquid hydrocarbons.

The present invention also provides a liquid hydrocarbon production method comprising
2a) reacting methane with H₂O and carbon dioxide to form syngas containing hydrogen and carbon monoxide,
2b) reacting the syngas to form liquid hydrocarbons, wherein the liquid hydrocarbon is alkane CₙH₂ₙ₊₂, where n=5-17, preferably n=5-10.

Further the present invention provides a liquid hydrocarbon production method comprising
3a) reacting methane with H₂O to form syngas containing hydrogen and carbon monoxide,
3b) reacting the syngas and carbon dioxide to form liquid hydrocarbons, wherein the liquid hydrocarbon is alkane CₙH₂ₙ₊₂, where n=5-17, preferably n=5-10.

In one aspect of the present invention the reactions 1a) and 3a) comprises steam reforming and the reaction 2a comprises combined steam reforming and CO₂ reforming.

In another aspect of the present invention the reaction 1b) comprises a Sabatier process.

In yet another aspect of the methods according to the present invention, the methods comprise supplying energy to one or more of the reactions.

In a further aspect the present invention provides methods according to the present invention, wherein the energy supplied is heat energy.

In another aspect the energy supplied is sustainable energy.

In one aspect of the methods the liquid hydrocarbon is alcohol CₙC₂ₙ₊₁OH, where n=1-20, preferably n=1-6.

In another aspect of the methods the liquid hydrocarbon is alkane CₙH₂ₙ₊₂, where n=5-17, preferably n=5-10.

In a further aspect of the methods according to the present invention the methods are oxygen-free such that no addition or formation of oxygen is required.

In yet another aspect of the methods according to the present invention, the methods comprises recycling heat energy obtained from reaction 1d), 2b) and 3b) respectively and or transformation of the obtained heat energy to electrical power. This energy could be used as input energy or sold as a bi-product.

The term “liquid” in connection with hydrocarbons, alkanes and alcohols as used herein refers to phase condition of the hydrocarbon at near atmospheric conditions. For alkanes the number of carbon atoms within the compound being between 5 and 17 which is equivalent to the number of carbon atoms being higher than or equal to five for the alkane to be described as liquid, whereas for alcohols also compounds with only one carbon atom such as methanol falls within the term liquid, typically alcohols are n=1-5. The method could also be used to produce other gas alkanes than CH₄ (n=2,3,4) or solid alkanes where n>=18.

The source of the carbon dioxide for the method can be any known CO₂ source such as CO₂ from reservoirs, CO₂ captured from industry or CO₂ captured from air, or combinations thereof.

The exothermic chemical reactions will be the main energy source but additional energy input may be required. Thermal energy can be utilized as the additional energy input. In an attractive embodiment sustainable energy is employed as the sole or main additional energy input. Other thermal energy sources could also be used; electricity input is also an option. Applicable energy sources include nuclear energy, or other type of energy (bio or fossil fuel)

The main principals of the present invention may be employed in the production of alkanes, alcohols and other liquid hydro carbons. The total reaction schemes for alkanes is

(n−1)/4CO₂+(3n+1)/4CH₄=>CₙH₂ₙ₊₂+(n−1)/2H₂O,

wherein n=alkane number

The total reaction schemes for alcohols is

n/4CO₂+(2−n)/2H₂O+3n/4CH₄=>CₙH₂ₙ₊₁OH,

wherein n=alcohol number.

As can be seen from the equation if the alcohol number is above 2 the amount of H₂O required on the left side of the equation becomes negative which is to be understood as water being produced:

n/4CO₂+3n/4CH₄=>CₙH₂ₙ₊₁OH−(2−n)/2H₂O,

Examples of specific total reactions are:

7CO₂+25CH₄=>4C₈H₁₈+14H₂O (Octane)

1CO₂+3CH₄=>2C₂H₅OH (Ethanol)

1CO₂+2H₂O+3CH₄=>4CH₃OH (Methanol)

As can be seen from all the equations no oxygen is added to the reactions, and no oxygen is therefore required to perform the method according to the present invention.

| | Example reactions | | |
|---|---|---|---|
| | Octane | Ethanol | Methanol |
| Traditional GTL | 14O₂ + 32CH₄ −> | 2O₂ + 4CH₄ −> | 1O₂ + 2CH₄ −> |
| | 4C₈H₁₈ + 28H₂O | 2C₂H₅OH + 2H₂O | 2CH₃OH |
| Liquid fuel from methane | 7CO₂ + 25CH₄ −> | 1CO₂ + 3CH₄ −> | 1CO₂ + 2H₂O + 3CH₄ −> |
| and CO₂ (RTL/GTL) | 4C₈H₁₈ + 14H₂O | 2C₂H₅OH | 4CH₃OH |
| % Production increase: | 28% | 33% | 33% |
| From “Traditional GTL” to | (4/32 −> 4/25) | (2/4 −> 2/3) | (2/2 −> 4/3) |
| “Liquid fuel from methane | | | |
| and CO₂” (per CH₄ used) | | | |

One or more of the following advantages can be obtained by the present invention:
Increased liquid fuel production per methane used compared to traditional GTL. The increase is theoretically 28-33%.
The present invention would have less CO₂-footprint than a traditional GTL process, some CO₂ is bound in the process.
Combustion of the obtained liquid hydrocarbon as fuel will have less CO₂-footprint than crude oil based fuels.
The process could utilize CO₂ from reservoirs, CO₂ captured from industry or CO₂ captured from air.
The present solution may have significant lower CAPEX and OPEX than traditional GTL-plants, due to that it avoids air gas plant to extract oxygen from air, and limits the need for gas power plants for generating electricity to run the processes, etc.
The present invention could be used as a renewable/nuclear energy export route. Periodically over-supply of renewable energy or nuclear energy can by this method be utilized to convert methane to liquid fuels; hence the renewable energy would be exported as “Renewable hydrocarbons”, CO₂-neutral liquid hydrocarbon fuels.

In a further aspect of the present invention the processes of production of alkanes and alcohols may be combined so that a combination of liquid alkanes and alcohols are obtained from methane and carbon dioxide.

### BRIEF DESCRIPTION OF THE DRAWINGS

The present invention will be exemplified in further detail with reference to the enclosed figures.

FIG. 1 illustrates a first embodiment for alkane production.

FIG. 2 illustrates a second embodiment for alcohol production.

FIG. 3 illustrates an alternative third embodiment for alkane production.

FIG. 4 illustrates a fourth embodiment for alcohol production.

FIG. 5 illustrates an alternative fifth embodiment for alkane production.

FIG. 6 illustrates an alternative sixth embodiment for alkane production.

FIG. 7 is a schematically illustration of the main principal of the present invention.

FIG. 8 illustrates the transfer of heat between inlet streams and product stream(s).

### PRINCIPAL DESCRIPTION OF THE INVENTION

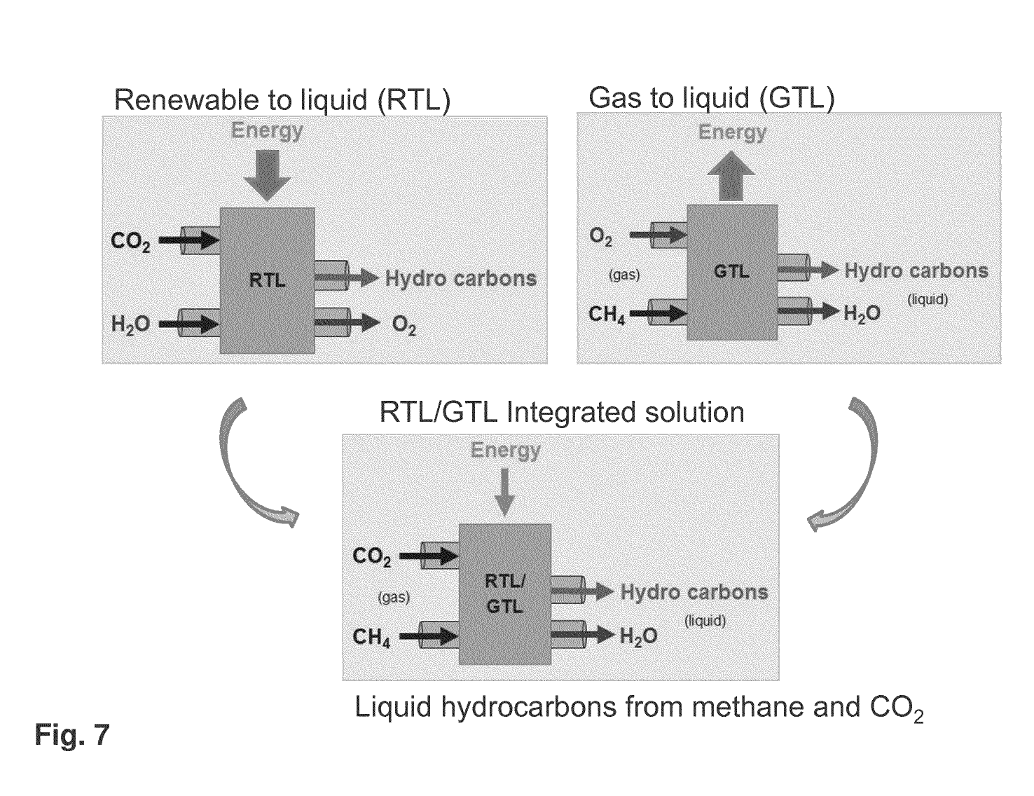

The main concept of the present invention is illustrated on FIG. 7. The present invention provides a combined integrated solution (RTL/GTL). In the known process of Renewable-to-Liquid (RTL) endothermic reactions are being employed or considered employed for the storage of renewable energy sources such as geothermal heat, sun light or wind energy resources. The renewable energy provides the energy to react H₂O with CO₂ to form hydrocarbons. This illustration is a simplification as the process is normally performed as a two step process, wherein energy is supplied to water to form hydrogen and oxygen and then in a second step the hydrogen is reacted with carbon dioxide to form hydrocarbons. This RTL process is in the present invention combined with a gas-to-liquid (GTL) process to form an integrated process.

The traditional GTL process comprises as illustrated here the reaction of methane with oxygen to form liquid hydrocarbons and water. The combined solution RTL/GTL according to the present invention comprises realisation of CO₂ as a valuable source of both oxygen and carbon and that the formation of and reaction with oxygen can be avoided by performing the reactions differently than by performing the RTL and GTL reactions in series, where oxygen produced in RTL is feed into a traditional GTL process.

The combined process has the potential of utilizing heat as renewable energy input and thereby provides a more cost efficient process.

The input energy is transmitted into the solutions as heat or power. This energy shall be used for the chemical reactions purposes. Rest heat in the in the produced outflowing chemicals (alkanes/alcohols, and H₂O if produced) may advantagesly be reclaimed by heat exchange systems. This heat is transferred into the inflowing chemicals (CH₄ and, CO₂, and H₂O when occurs). To secure limited energy leakage, insulation can be provided around all processes with high temperature. This construction will make the solutions very energy efficient. By this the input energy will efficiently be used to fill the gap between the chemical energy potential in produced alkanes or alcohols and the chemical energy potential in the inflowing CH₄. For some of the reactions, alkanes of higher order, the inflowing CH₄ will have higher chemical energy potential than the produced alkanes. In these situations surplus heat will be lead away with the outflowing materials.

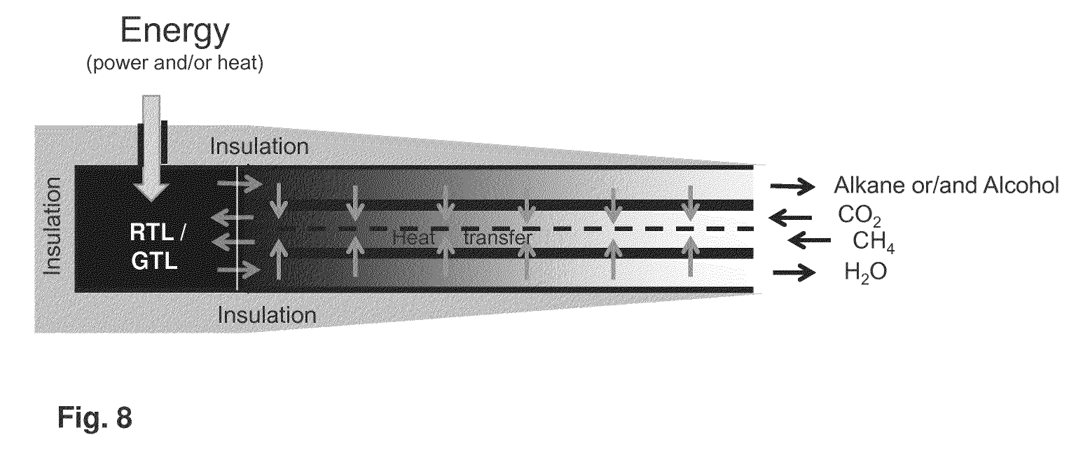

FIG. 8 shows one set up of such combination of insulation and heat transfer from outflowing to inflowing chemicals. Pipe-in-pipe solution with countercurrent flow ensures a heat gradient that allows heat to go from outflowing chemicals to inflowing. Theoretically if all heat is regained the energy input needed or the heat energy produced is determined by the energy produced and released by the chemical reactions.

The following table shows the overall difference in enthalpy for three examples of the combined RTL/GTL reactions according to the present invention.

| | EXAMPLE REACTIONS | | |
|---|---|---|---|
| | OCTANE | ETHANOL | METHANOL |
| Liquid fuel | 7CO₂ + 25CH₄ −> | 1CO₂ + | 1CO₂ + |
| from natural | 4C₈H₁₈ + 14H₂O | 3CH₄ −> | 2H₂O + |
| gas and CO2 | | 2C₂H₅OH | 3CH₄ −> |
| (RTL/GTL) | | | 4CH₃OH |
| ΔH (kJ/mole) - | −94 | 31 | 59 |
| per mole HC | | | |
| molecule | | | |

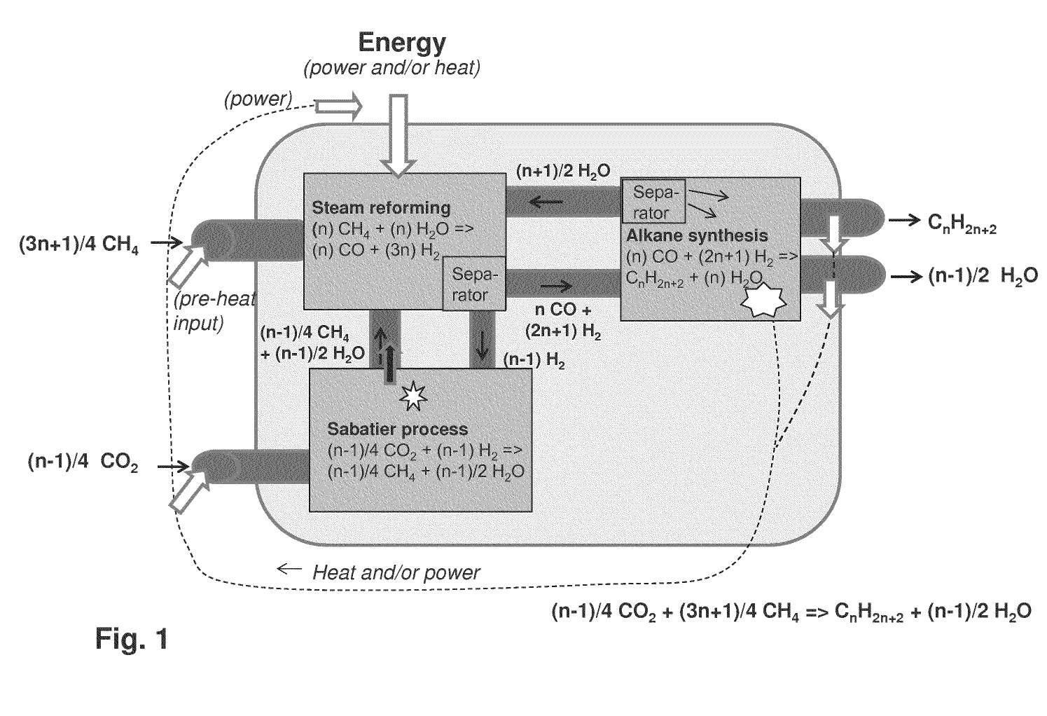

The energy efficiency of the conversion is enforced by insulation around the converter and heat transfer from outflowing products to inflowing material streams, by use of countercurrent pipe-in-pipe system as illustrated in FIG. 8, or any other methods of transferring heat. Heat based power generation could be built as part of this heat transfer from warm to cold product streams. This power generation is marked as star in the exothermic processes in the FIGS. 1 to 6. This power could be used as input to endothermic processes.

FIG. 1 illustrates a first embodiment of the present invention for the production of alkanes. Here the process is split into three reactions, steam reforming, Sabatier process and alkane synthesis. Each of these steps are in them self known reaction processes but the integrated combination as disclosed is new.

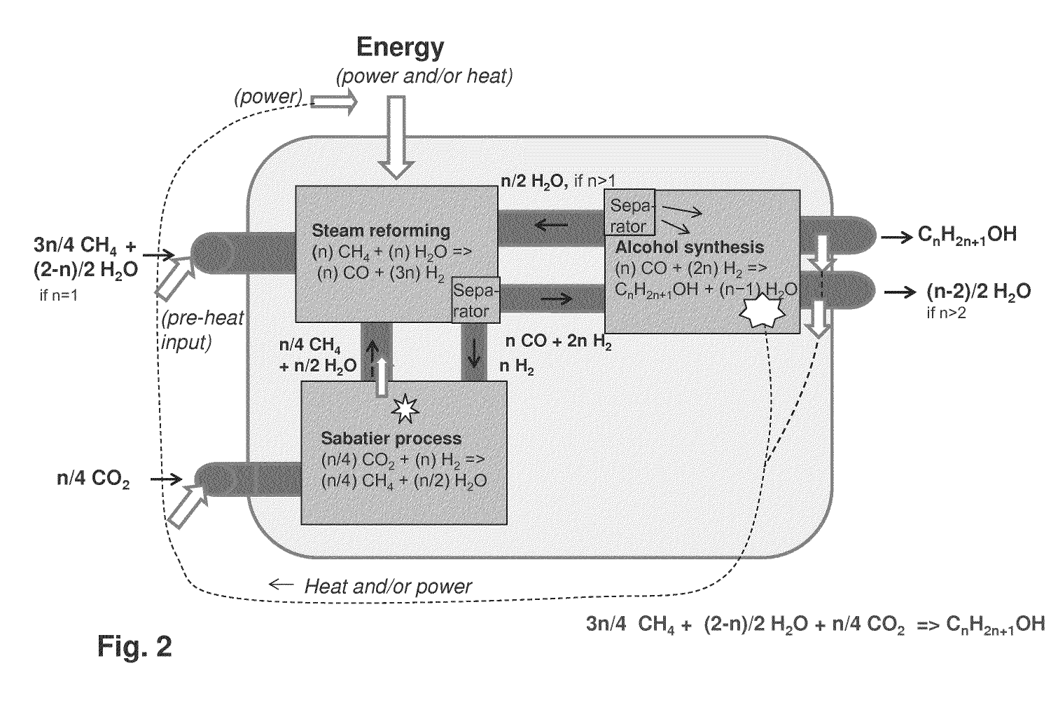

In the steam reforming step methane gas is reacted with water under increased temperature and in the presence of a catalyst to form syngas comprising carbon monoxide and hydrogen. Steam reforming can be performed at different conditions and the present invention is not limited to any of these known methods. A part of the obtained hydrogen is separated from the syngas and transferred to the Sabatier process. Here the hydrogen is reacted with carbon dioxide to form methane and water. The Sabatier process as such is known. The reaction products are transferred to the steam reforming to form part of the raw material for this process. The remaining syngas comprising a reduced amount of hydrogen is transferred to the alkane synthesis resulting in liquid alkanes, which will be higher alkanes comprising more than one carbon atom and water. A part of the produced water is separated and transferred back to the steam reforming to supply the water needed for this process. Taken as a whole the inlet streams are methane and CO₂ and the outlet streams are liquid alkanes CₙH₂ₙ₊₂ where n=5-17 and water. The energy consumption and production is also illustrated in FIG. 2 by the fat arrows and the stars. Energy is added to the steam reforming to provide the heat for the process. This heat can at least partly be supplied by pre-heating the methane with surplus of energy from the exothermic alkane synthesis or by heat transfer with the steam reforming. Heat is also transferred with the H₂O from the alkane synthesis and the gasses supplied from the exothermic Sabatier process.

FIG. 2 illustrate a second embodiment of the present invention which differs from the embodiment of FIG. 2 only in that the liquid hydrocarbon formed by the overall process is an alcohol CₙH₂ₙ₊₁OH, where n>=1, preferably n=1-20, more preferably n=1-10. The alcohol synthesis results in formation of less H₂O, and all the H₂O can therefore be recycled to the steam reforming, however that will not supply sufficient water and therefor additional water has to be supplied to the steam reformer. The ration between the carbon inlet streams CH₄ and CO₂ is also different in the two embodiments as a higher ratio of CO₂ can be transformed in the alcohol process.

In a further embodiment of the present invention the processes of the first and the second embodiment may be combined so that a combination of liquid alkanes and alcohols are obtained from methane and carbon dioxide.

The total reactions of embodiment 1 and 2:

(n)CH₄+(n)H₂)=>(n)CO+(3n)H₂

(n−1)/4CO₂+(n−1)H₂=>(n−1)/4CH₄+(n−1)/2H₂O

(n)CO+(2n+1)H₂=>CₙH₂ₙ₊₂+nH₂O Alcohol production (1):

(n)CH₄+(n)H₂O=>(n)CO+(3n)H₂

(n/4)CO₂+(n)H₂=>(n/4)CH₄+(n/2)H₂O

(n)CO+(2n)H₂=>CₙH₂ₙ₊₁OH+(n−1)H₂O Alcohol production (2):

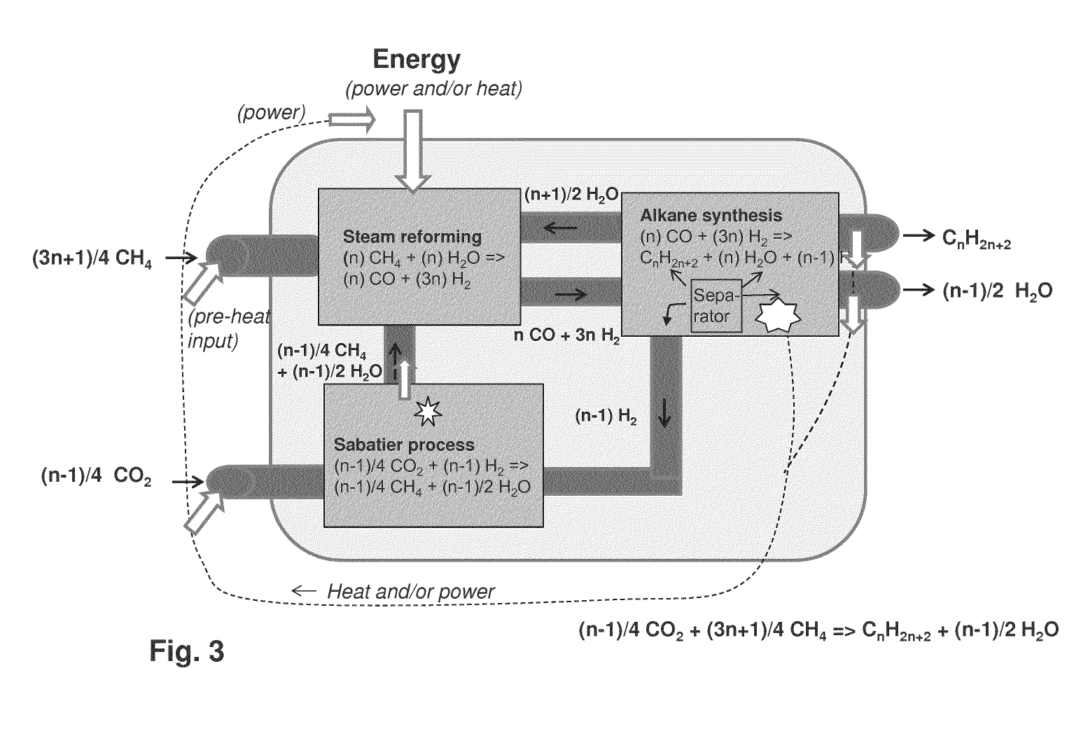

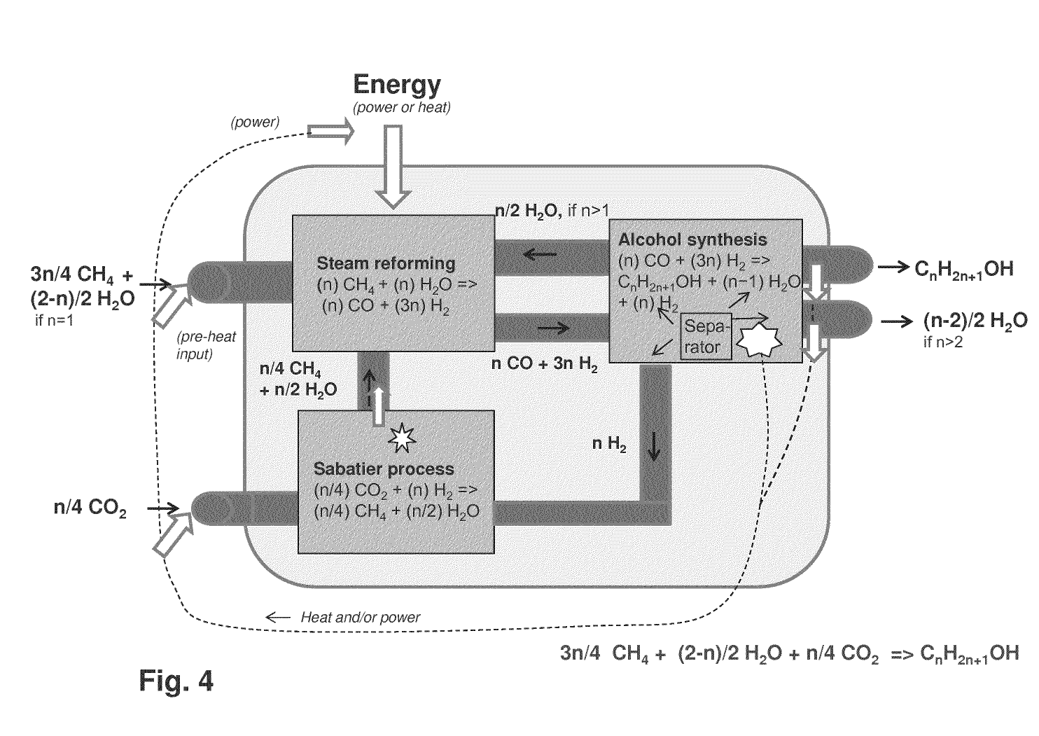

FIG. 3 illustrates an alternative third embodiment of the present invention. Here the present invention is exemplified by the production of alkanes but compared to the embodiment illustrated on FIG. 2 the embodiment on FIG. 4 requires less separators as the separation of hydrogen from the syngas is performed together with the separation of water after the alkane synthesis step.

FIG. 4 illustrates a fourth embodiment similar to the third embodiment but for alcohol synthesis and is comparable with FIG. 3. In this embodiment the separation of a part of the produced hydrogen within the syngas is separated out from or after the alcohol synthesis and accordingly no separation is needed as part of the steam reforming.

When the separation of the hydrogen for the Sabatier process is performed after the formation of liquid hydrocarbons the separation process could be based on phase separation between liquids and hydrogen gas.

The total reactions of embodiment 3 and 4:

(n)CH₄+(n)H₂O=>(n)CO+(3n)H₂

(n−1)/4CO₂+(n−1)H₂=>(n−1)/4CH₄+(n−1)/2H₂O

(n)CO+(3n)H₂=>CₙH₂ₙ₊₂+(n)H₂O+(n−1)H₂ Alkane production (3):

(n)CH₄+(n)H₂O=>(n)CO+(3n)H₂

(n/4)CO₂+(n)H₂=>(n/4)CH₄+(n/2)H₂O

(n)CO+(3n)H₂=>CₙH₂ₙ₊₁OH+(n−1)H₂O+(n)H₂ Alcohol production (4):

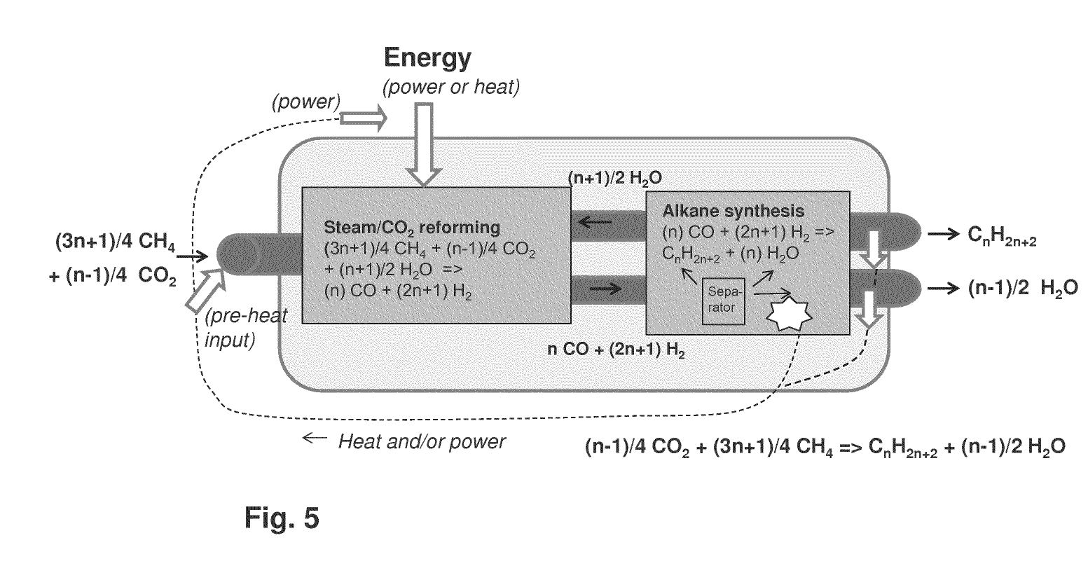

FIG. 5 illustrates a further embodiment of the present invention comprising a combined reforming of methane, H₂O and CO₂ and production of alkane.

The total reactions of embodiment 5:

(3n+1)/4CH₄+(n−1)/4CO₂+(n+1)/2H₂O=>nCO+(2n+1)H₂

nCO+(2n+1)H₂=>CₙH₂ₙ₊₂+nH₂O Alkane production (5)

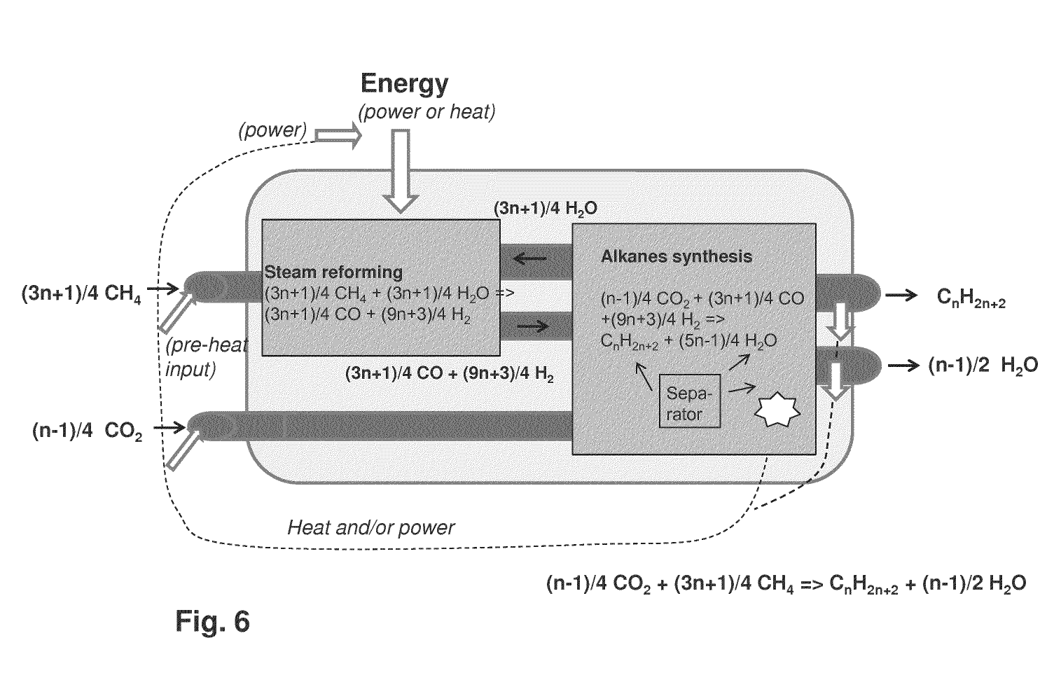

FIGS. 6 illustrates a further embodiment of the present invention comprising a steam reforming of methane and H₂O and production of alkane. In this embodiment CO₂ is added to the last step of the processes comprising formation of alkane.

The total reactions of embodiment 6:

(3n+1)/4CH₄+(3n+1)/4H₂O=>(3n+1)/4CO+(9n+3)/4H₂

(n−1)/4CO₂+(3n+1)/4CO+(9n+3)/4H₂=>CₙH₂ₙ₊₂+(5n−1)/4H₂O Alkane production (6)

1. Liquid hydrocarbon production method comprising
1a) reacting methane with H₂O to form syngas containing hydrogen and carbon monoxide,
1b) reacting a part of the hydrogen with carbon dioxide to form methane and H₂O,
1c) supplying said methane and H₂O obtained from carbon dioxide to the syngas forming reaction and
1d) reacting the remaining syngas to form liquid hydrocarbons.

2. Liquid hydrocarbon production method comprising
2a) reacting methane with H₂O and carbon dioxide to form syngas containing hydrogen and carbon monoxide,
2b) reacting the syngas to form liquid hydrocarbons, wherein the liquid hydrocarbon is alkane CₙH₂ₙ₊₂, where n=5-17, preferably n=5-10.

3. Liquid hydrocarbon production method comprising
3a) reacting methane with H₂O to form syngas containing hydrogen and carbon monoxide,
3b) reacting the syngas and carbon dioxide to form liquid hydrocarbons, wherein the liquid hydrocarbon is alkane CₙH₂ₙ₊₂, where n=5-17, preferably n=5-10.

4. Method according to claim 1, wherein the reaction 1a) and 3a) comprises steam reforming and the reaction 2a) comprises combined steam reforming and CO₂ reforming.

5. Method according to claim 1, wherein reaction 1b) comprises a Sabatier process.

6. Method according to claim 1, comprising supplying energy to one or more of the reactions.

7. Method according to claim 6, wherein the energy supplied is heat energy.

8. Method according to claim 6 wherein the energy supplied is sustainable energy.

9. Method according to claim 1, wherein the liquid hydrocarbon is alcohol CₙH₂ₙ₊₁OH, where n=1-20, preferably n=1-6.

10. Method according to claim 1, wherein the liquid hydrocarbon is alkane CₙH₂ₙ₊₂, where n=5-17, preferably n=5-10.

11. Method according to claim 1, wherein the method is oxygen-free such that no addition or formation of oxygen is required.

12. Method according to claim 1, wherein the method comprises recycling heat energy obtained from reaction 1d), 2b) or 3b) respectively and or transformation of the obtained heat energy to electrical power.
